(19) United States
(12) Patent Application Publication
(10) Pub. No.: US 2007/0009438 A1
(43) Pub. Date: Jan. 11, 2007

(54) Method for diagnosing diabetes type 2 using standardized mixed meal and calculated index values

(75) Inventor: Wilfred Wayne Lautt, Winnipeg (CA)

Correspondence Address: MERCHANT & GOULD PC, P.O. BOX 2903, MINNEAPOLIS, MN, 55402-0903, US

(73) Assignee: DiaMedica Inc., Winnipeg (CA)

(21) Appl. No.: 10/569,248

(22) PCT Filed: Aug. 5, 2004
(86) PCT No.: PCT/CA04/01463

§ 371 Date: Sep. 15, 2006

(30) Foreign Application Priority Data
Aug. 26, 2003 (US) 60497553

(51) Int. Cl. A61K 49/00; C12Q 1/54; G06F 19/00

(52) U.S. Cl. 424/9.1; 435/14; 702/19

(57) ABSTRACT

The present invention provides methods and materials for diagnosing a metabolic disorder comprising measuring insulin and HISS action or alternatively HISS levels, first in a fasting state and again, following consumption of a mixed standard meal. The metabolic disorders diagnosed using the disclosed methods and materials include insulin resistance, pre-diabetes and diabetes. The present invention also provides kits for practicing the disclosed methods.

### FIELD OF INVENTION

The present invention relates to products and methods for diagnosing metabolic disorders, and more specifically insulin resistance, pre-diabetes, and diabetes.

### BACKGROUND

The present inventor has recently discovered a novel hepatic mechanism of regulating insulin sensitivity wherein postprandial elevation in insulin levels activate a hepatic parasympathetic nerve-dependent release of the hormone, hepatic insulin-sensitizing substance (HISS), which activates glucose uptake in skeletal muscle.

The present inventor discovered that feeding leads to the activation of hepatic parasympathetic permissive signal acting through release of acetylcholine which leads to generation of nitric oxide. Insulin in response to this permissive signal results in the release of HISS. HISS sensitizes skeletal muscle to the action of insulin (or has a direct insulin-like action). The response to insulin consists of a quite constant HISS-independent component and a larger, but highly regulated, HISS-dependent component that is greatest in the immediate postprandial state and least in the fasted state, when insulin action is normally neither required nor desirable.

Interruption of HISS release by surgical or pharmacological ablation leads to immediate severe insulin resistance. Hepatic denervation-induced insulin resistance is reversible by provision of intraportal venous acetylcholine or a nitric oxide donor at the time of administration of insulin. Hepatic parasympathetic neuropathy results in the type of metabolic disturbance that occurs in patients with type 2 diabetes (non-insulin-dependent), liver disease, obesity, and essential hypertension. All of these diseases show associated insulin resistance and parasympathetic neuropathy which appears to be causal, with the neuropathy causing HISS-dependent insulin resistance (HDIR).

As shown in FIG. 1, following a meal, a feeding signal is transmitted to the liver by the hepatic parasympathetic nerves. This permissive signal and the subsequent release of HISS effectively doubles the glucose storage effect of insulin. HISS acts selectively on skeletal muscle to stimulate glucose uptake and storage as glycogen. This accounts for the observation that the majority of glucose absorbed from a meal is stored as glycogen in skeletal muscle. HISS accounts for approximately 55% of the total glucose disposal caused by the administration of insulin. Interruption of the parasympathetic signal results in blockade of HISS release and a state of HDIR. The condition of HDIR results in postprandial hyperglycemia, hyperinsulinemia, and hyperlipidemia.

Currently, there are two principle methods of diagnosing diabetes: (1) measuring fasting plasma glucose concentrations and (2) measuring post-load plasma glucose concentrations using the oral glucose tolerance test. Both tests are considered suitable for the diagnosis of diabetes by the American Diabetes Association. However, the fasting plasma glucose concentration test is considered preferable because it is easier and faster to perform, more convenient and acceptable to patients, less expensive, and produces more reproducible results.

Test meals, both solid and liquid have been used to test for the ability to release insulin and for the change in blood glucose occurring in response to the meal. The standard use of a test meal provides results which are indistinguishable from the results obtained using the oral glucose tolerance test. In a study designed specifically to show the superiority of the test meal method compared to the oral glucose tolerance test, the major claim for the test meal was a somewhat reduced variability in the blood glucose levels attained using the test meal. However, the observed differences in variability were minor and likely the result of differences in the glucose load administered with the oral glucose being 50% higher (75 grams) than that administered by the test meal (50 grams). As the glycemic load is increased, the physiological mechanisms to cope are more stressed thereby leading to a greater variability. Regardless of this explanation, it has been argued that the two tests are equal in diagnostic value for both detecting glucose intolerance and diabetes (Wolever et al., 1998). However, as referred to earlier, the American Diabetes Association clinical practice recommendations indicate that fasting glucose concentration test is equal and preferable to the oral glucose tolerance test and that the repeat test reproducibility of the oral glucose tolerance test is worse than that of the fasting plasma glucose method.

The American Diabetes Association has noted that as many as 50% of people with diabetes are undiagnosed. The Association has noted that many subjects diagnosed with diabetes have evidence of retinopathy, a condition which may begin to develop at least seven years before a clinical diagnosis of type 2 diabetes is made (Diabetes Care, January 2003). This indicates that the standard means of diagnosing this disease are only capable of detecting the disease at an advanced stage when there are abnormalities in fasting plasma glucose concentrations or the response to oral glucose is impaired. By the time abnormalities in the fasted state are detectable, the disease has already progressed well along. This suggests that the major problem that occurs in diabetes is with the processing of a meal and not in the fasted stated.

Recently, it has been discovered that the administration of glucose alone to rats, rather than a solid or liquid mixed meal, fails to elicit the release of HISS. It appears that glucose alone is either incapable of serving as the feeding signal or the response is sufficiently transient to be essentially eliminated by the time of testing. Currently, there are no clinical tests for diagnosing HDIR.

Earlier detection of diabetes will allow earlier control of blood glucose levels and a reduction in the severity of complications associated with this disease.

Accordingly, there is a need for a test which can diagnose insulin resistance and pre-diabetes, prior to progression to full diabetes. Specifically, there is a need to assess the ability to respond to a meal in an appropriate manner and to diagnose HDIR.

### SUMMARY OF THE INVENTION

The present invention provides a method of diagnosing a metabolic disorder comprising the steps of: determining a first index value in a fasted test subject, wherein the first index value correlates with insulin action; feeding the test subject a standardized mixed meal; determining a second index value in the fed test subject, wherein the second index value correlates with insulin and HISS action; calculating a third index value using the first and second index values wherein the third index value correlates with HISS action; and comparing the third index value with a reference value range wherein a metabolic disorder is diagnosed if the third index value is outside of the reference value range.

The metabolic disorder to be diagnosed includes insulin resistance, pre-diabetes and diabetes.

In an embodiment of the present invention, the first index value is determined by: taking blood samples from a fasting test subject and measuring glucose concentrations at regular intervals until the measured glucose concentrations are substantially stable; taking blood samples from the insulin infused test subject and measuring the test subject's glucose concentrations at intervals; infusing the test subject with glucose and adjusting the glucose quantity to maintain a stable glucose level, at baseline, pre-insulin levels; and determining the amount of glucose necessary to establish euglycemia in the test subject wherein the amount of glucose establishes the first index value.

In another embodiment of the present invention, the second index value is determined by: taking blood samples from the insulin infused test subject and measuring the test subject's glucose concentrations at intervals; infusing the test subject with glucose and adjusting the glucose quantity to maintain a stable glucose level, at baseline, pre-insulin levels; and determining the amount of glucose necessary to establish euglycemia in the test subject wherein the amount of glucose establishes the second index value.

In another embodiment of the present invention, the first index value is determined by: taking blood samples from a fasting test subject and measuring glucose concentrations at regular intervals until the measured glucose concentrations are substantially stable; infusing the test subject with a predetermined quantity of insulin; taking blood samples from the insulin infused test subject and measuring glucose concentrations at regular intervals until the measured glucose concentrations are substantially stable; and determining a nadir value in the measured glucose concentrations of the insulin infused test subject wherein the nadir value establishes the first index value.

In another embodiment of the invention, the second index value is determined by: taking blood samples from the test subject and measuring glucose concentrations at regular intervals until the measured glucose concentrations are substantially stable; infusing the test subject with a predetermined quantity of insulin; taking blood samples from the insulin infused test subject and measuring glucose concentrations at regular intervals until the measured glucose concentrations are substantially stable; determining a nadir value in the measured glucose concentrations of the insulin infused test subject wherein the nadir value establishes the second index value.

In another embodiment of the present invention, the second index value is determined by: taking blood samples from the test subject and measuring glucose concentrations at regular intervals until the measured glucose concentrations are substantially stable; infusing the test subject with a predetermined quantity of insulin; waiting 15 minutes and then taking a blood sample from the insulin infused test subject and measuring glucose concentrations wherein the measured glucose concentration establishes said second index value.

In another embodiment of the present invention, the standardized mixed meal comprises carbohydrate, protein and fat.

The present invention also provides a method of diagnosing a metabolic disorder comprising the steps of: measuring glucose concentration and HISS concentration in a fasted test subject to provide respectively a first glucose value and a first HISS value; feeding the test subject a standardized test meal; measuring glucose concentration and HISS concentration in the fed test subject to provide respectively a second glucose value and a second HISS value; calculating a third glucose value using the first glucose value and the second glucose value; calculating a third HISS value using the first HISS value and the second HISS value; comparing the third glucose value with a reference glucose value; and comparing the third HISS value with a reference HISS value, wherein a metabolic disorder is diagnosed if the third glucose value and the third HISS value are outside of the reference glucose value range and the reference HISS value range respectively.

The present invention also provides a kit for use in diagnosing a metabolic disorder comprising a pre-measured quantity of a standardized mixed meal and instructions.

### BRIEF DESCRIPTION OF THE FIGURES

FIG. 1 shows the relationship between glucose, insulin and HISS and the respectively physiological effects.

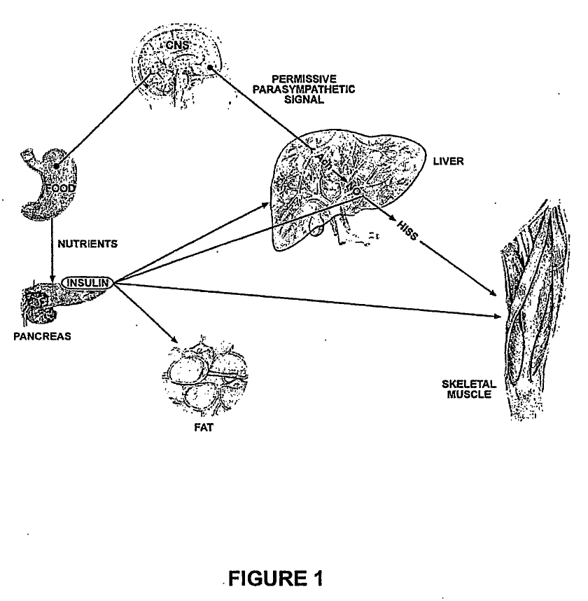

FIG. 2 is a bar graph showing the effect of HISS action on insulin sensitivity in rats under varying feeding conditions.

FIG. 3 is a bar graph showing insulin sensitivity in rats before and after feeding.

FIG. 4 is a bar graph showing insulin sensitivity in rats fed the liquid test meal of the present invention versus rats fed glucose.

FIG. 5 is a bar graph showing insulin sensitivity in humans fed the liquid test meal of the present invention.

FIG. 6 is a graph comparing HISS response and glucose load in humans.

### DETAILED DESCRIPTION

The present invention is based on the discovery that insulin, in the presence of a permissive hepatic parasympathetic nerve signal in the liver, results in the release in HISS and ultimately the uptake of glucose in skeletal muscle. The present invention is further based on the discovery that glucose alone is incapable of eliciting HISS release in a diagnostically useful manner whereas a mix meal does provide a sufficient feeding signal to allow HISS release. Accordingly, the detection of HISS release can be determined either by measuring insulin and HISS action, or far less invasively, by simply measuring HISS levels before and after the consumption of a mixed meal. The change in glucose and HISS levels in response to the test meal is the diagnostic index.

The present invention provides a method of diagnosing a metabolic disorder by examining insulin action in a test subject after fasting and again, after the consumption of a standardized mixed meal. The metabolic disorder diagnosed using this method includes insulin resistance, pre-diabetes and diabetes. The method may also be used for other metabolic disorders for which HISS dependent insulin resistance is a causal factor. This method has the advantage of being able to detect pre-diabetic conditions which cannot be detected using the conventional fasting glucose and oral glucose tolerance tests.

The method comprises four major steps: determining a first index value in a fasted test subject where the first index value correlates with insulin action; feeding the test subject a standardized mixed meal; determining a second index value in the fed test subject, where the second index value correlates with insulin and HISS action; calculating a third index value using the first and second index values where the third index value correlates with HISS action; and comparing the third index value with a reference value corresponding to normal insulin and HISS function.

Insulin and HISS action can be measured using either the insulin tolerance test or the rapid insulin sensitivity test. Typically, the test subject will fast overnight for 16-24 hours prior to measurement of insulin and HISS action by either test.

In a preferred embodiment of the invention, the rapid insulin sensitivity test is used. The rapid insulin sensitivity test is advantageous over the insulin tolerance test as it allows for multiple testing in the same day while also avoiding hypoglycemia which causes subject discomfort and the activation of counterregulatory hormones which act to prevent prolonged decline of blood glucose levels in response to insulin.

The first step in determining the first index value is to establish a reference glycemic baseline for the fasting test subject. This is accomplished by measuring arterialized venous glucose concentrations at regular intervals until the glucose concentrations reach a stable plateau. Arterialized venous blood samples can be obtained using methods known in the art. A catheter can be inserted into the test subject to allow for ease of multiple sampling. Preferably, glucose concentrations will be checked at 5 minute intervals until three consecutive concentrations are observed which are substantially similar. Blood glucose can be measured using methods known in the art such as the oxidase method wherein glucose is oxidized in the presence of glucose oxidase and the amount of glucose measured using a glucose analyzer (Yellow Springs Instrument Co., Yellow Springs, Ohio). Other known methods for accurately quantitating blood glucose concentrations can also be used.

Once a stable glycemic baseline is established, the next step in determining the first index value is to infuse the test subject with insulin. This can be accomplished using an infusion pump. The amount of insulin used will depend on the species of the test subject. A person skilled in the art can determine the necessary amount of insulin to elicit glucose uptake by titrating the amount of insulin administered and measuring glucose concentrations. Preferably, the amount of insulin used will elicit a measurable change in glucose uptake. For a human test subject, the amount of insulin used will preferably be 50 mu/kg.

Following insulin infusion, generally after 2 minutes, the test subject is then variably infused with glucose. Glucose may be administered as a glucose-saline solution using an infusion pump. In a preferred embodiment, a 10% glucose solution is administered. The test subject's arterialized venous glucose concentration is then determined at regular intervals and the rate of glucose infusion adjusted until the measured glucose concentrations maintain a stable plateau representing euglycemia. Preferably, glucose concentrations are measured at 2 minute intervals.

The first index value is determined by measuring the amount of glucose necessary to maintain euglycemia. The first index value is expressed in terms of milligrams of glucose per kilogram of body weight and is a measure of insulin action.

Once the first index value is determined using the rapid insulin sensitivity test, the test subject is fed a standardized mixed meal to elicit HISS mediated insulin action. The standardized mixed meal is comprised of a mixture of carbohydrate, protein and fat. The amount of each individual component will depend on the species of the test subject. For rats, an individual meal portion will preferably contain 0.1 gram of carbohydrates. For humans, an individual meal portion can contain varying proportions of lipid and protein, any may contain 25 to 100 grams of carbohydrate and even more preferably, 50 grams of carbohydrates. The standardized mixed meal may either be solid or liquid form and may contain ingredients such as flavourings and additives to make the product more palatable and to extend the meal's shelf-life.

Shortly after the standardized mixed meal is consumed, the second index value can be determined using the same procedures as described for the determination of the first index value. The second rapid insulin sensitivity test is carried out after a new stable glycemic baseline has been obtained, typically 90 minutes after consumption of the meal. The second index value represents the combined action of insulin and HISS.

While the rapid insulin sensitivity test is preferable for the determination of the first and second index values, the insulin tolerance test can also be employed.

To determine the first index value using the insulin tolerance test, the first step is to establish a reference glycemic baseline. The same procedure as described above in relation to the rapid insulin sensitivity test can be employed. Once the glycemic baseline is established, the test subject is infused with insulin, also as described above. Glucose levels are measured to assess the hypoglycemic response to the insulin before counterregulatory mechanisms are activated. The second index value can be determined by measuring glucose samples at regular intervals, and more preferably at 2 minute intervals, until the measured glucose concentrations reach a nadir (the lowest observed glucose concentration). Since in humans, the nadir is generally observed within 15 minutes of insulin administration, the second index value can also be determined by taking a single blood sample at this point. After the second index value is determined, hypoglycemia and the associated subject discomfort can be terminated by administrating glucose.

Once the first and second index values are determined using either the rapid insulin sensitivity test or the insulin tolerance test, a third index value which correlates with HISS action, can be determined and compared with established reference values in order to diagnose the test subject. Generally, the third index value is calculated by determining the difference between the first and second index values. Where the rapid insulin sensitivity test is used to determine the first and second index values, the third index value can be expressed in terms of: (1) the absolute increase in insulin action, and (2) the proportion of insulin action mediated by HISS.

The diagnostic method described above provides for an extremely accurate method for diagnosing insulin resistance, pre-diabetes and diabetes. However, because multiple blood samples are required, the method is not ideal for mass screening. The present invention provides an alternate method for diagnosing insulin resistance, pre-diabetes and diabetes which requires only the minimum of blood samples.

This method involves taking a blood sample from a fasting test subject and measuring the glucose concentration and the HISS concentration to determine respectively, a first glucose value and a first HISS value. The blood sample can be obtained by venous puncture or by finger prick. The test subject is then administered the standardized mixed meal as previously described. Following consumption of the test meal, generally after 20-90 minutes, a second glucose value and a second HISS value are determined.

Glucose concentrations can be measured using known methods such as the oxidase method described above. Widely available blood glucose monitors such as Accu-Chek™ (Roche Diagnostics) and OneTouch™ (Lifescan) can also be used. HISS concentrations can be measured using a variety of methods known in the art. Illustrative methods include, but are not limited to, chromatographic methods (e.g., high performance liquid chromatography, gas chromatography), spectrometric methods (e.g. mass spectrometry) and immunoassays (e.g. enzyme-linked immunosorbent assay). The increase in glucose concentration—the third glucose value—is calculated by subtracting the first glucose value from the second glucose value. Similarly, the increase in HISS concentrations—the third HISS value—is calculated by subtracting the first HISS value from the second HISS value. The third glucose value and the third HISS value are then compared to references values in order to make a diagnosis. In a subject with frank diabetes, a large elevation in blood glucose level and a small or no elevation in HISS would be observed. The pre-diabetic, where current diagnosis is inadequate would show responses between these extremes with HDIR being detectable at earlier time points than are determined using classical fasting glucose levels, oral glucose tolerance test, or test meals where HISS levels or HISS action is not determined

The present invention also provides kits for use in diagnosing HISS dependent insulin resistance which is applicable for practicing the methods of the present invention.

In one embodiment of the present invention, a kit is provided comprising a premeasured quantity of a standardized test meal and instructions for use in measuring insulin and HISS action. The kit may further comprise a premeasured quantity of insulin and a premeasured quantity of glucose for use in performing the rapid insulin sensitivity test.

In another embodiment of the present invention, a kit is provided for use in measuring glucose and HISS concentrations and comprises a premeasured quantity of a standardized mixed meal and instructions for use. The kit may further comprise a device for measuring blood glucose concentrations such as a glucose monitor and test strips. The kit may also further comprise an assay for use in measuring HISS concentrations. Where the assay is an immunoassay, the kit may further comprise an antibody specific for compounds of the invention.

In a further embodiment, the invention provides a kit supplying a calibrated liquid meal to be self administered. The kit includes a glucose measuring device, for example test strips or a glucose meter, alcohol swabs and lancet. Blood glucose is determined using a finger prick in the fasted state and again 90 minutes after consumption of the test meal. The rise in glucose will correlate negatively with the rise in HISS. HISS chemical determination can also be performed.

It is to be understood that while the invention has been described in conjunction with the preferred specific embodiments thereof, that the description above as well as the examples which follow, are intended to illustrate and not limit the scope of the invention. Other aspects, advantages and modifications within the scope of the invention will be apparent to those skilled in the art to which the invention pertains.

### EXAMPLE ONE—QUANTITATION OF HISS ACTION IN RATS

Male Sprague-Dawley rats were catherized and divided into 3 groups and fasted for 6, 18 or 24 hours. The rats were then re-fed with standard rat chow ad libitium. A RIST was then performed by infusing each rat with a 50 mU·kg⁻¹ dose of insulin over a 5 minute period (0.5 mL volume at 0.1 mL·min-1) using an infusion pump (Harvard Apparatus, Millis, Mass.). After 1 minute of infusion, an arterial blood sample was taken to assess blood glucose and a variable glucose infusion (10%) was initiated. Blood samples were then taken every 2 minutes and the glucose infusion rate adjusted to maintain euglycemia. The RIST index is the amount of glucose infused, to maintain euglycemia over the test period that terminated when no further glucose infusion was required (approximately 30-35 min).

The rats were then treated with atropine (3 mg·kg⁻¹ i.v.) to block HISS release and a second RIST was performed.

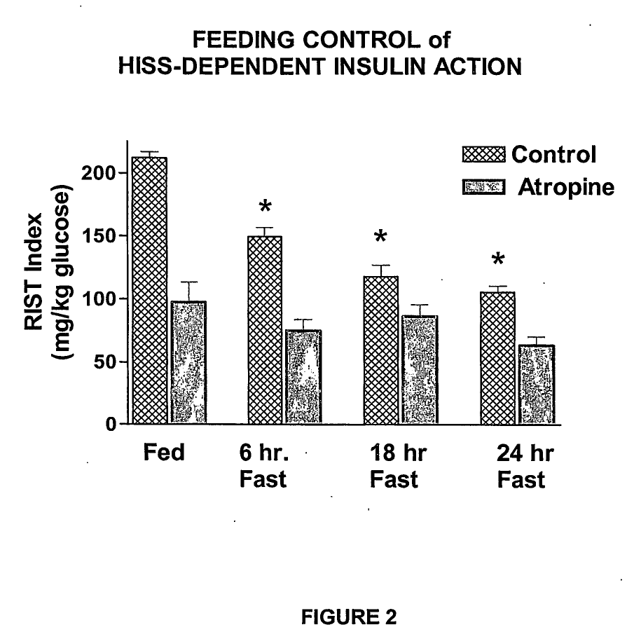

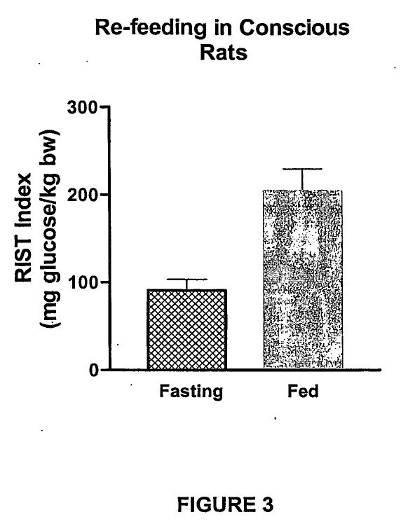

As shown in FIG. 2, the glucose disposal effect of an injection of insulin progressively decreased with the duration of fasting. A 24-hour fast in rats resulted in a RIST index not significantly different from that seen after atropine (which blocks HISS release) thereby indicating that the dramatic decrease in insulin action seen with fasting is due to the decline in HISS action with no significant alteration in the HISS-independent component of insulin action seen after atropine. Upon re-feeding, insulin action is increased as shown in FIG. 3.

### EXAMPLE TWO: DEVELOPMENT AND VALIDATION OF TEST MEAL TO QUANTITATE HISS ACTION IN RATS

In a fed animal, use of the RIST demonstrates a high sensitivity to insulin that can be reduced by 55% by blocking HISS release. A 24-hour fast in rats results in a RIST index not significantly different from that seen after atropine (which blocks HISS release), thereby indicating that the dramatic decrease in insulin action seen with fasting is due to the decline in HISS action with no significant alteration in the HISS-independent component of insulin action seen after atropine (FIG. 2). Upon re-feeding, insulin action is increased (FIG. 3) (Latour and Lautt, 2002). These observations led to the attempt to utilize a standardized test meal to diagnose the ability of insulin to cause the release of HISS and thereby serve as a method for diagnosing HDIR. This approach proved unfeasible for development in animals owing to the inability of the rats to consistently consume a standard volume of food within a standard period of time. To obviate this problem, the use of a liquid test meal was examined.

Male Sprague-Dawley rats were catherized, fasted overnight and divided into two test groups: test meal and glucose.

The test meal rats were administered a 2.5 ml gavage of a liquid mixed meal containing 0.1 g protein, 0.06 g fat, and 0.4 g carbohydrate. After one hour, the test meal rats were anesthetized, instrumented and submitted to a RIST as described in Example One.

The glucose rats were administered a 2.5 ml gavage containing 0.5 g of glucose. After one hour, the glucose rats were anesthetized, instrumented and submitted to a RIST as described in Example One. The glucose rats were then treated with atropine (3 mg·kg⁻¹ i.v.) to block HISS release and a second RIST was performed.

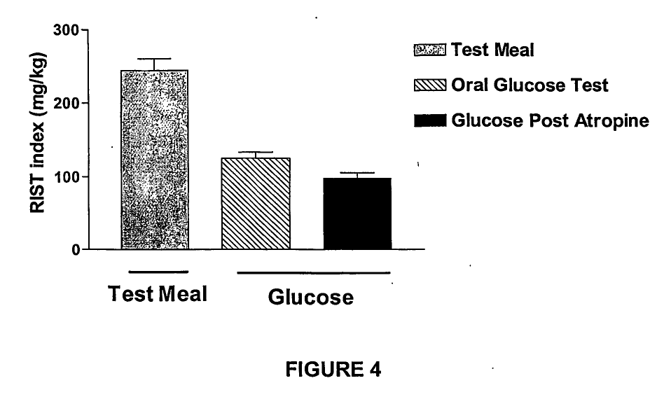

As shown in FIG. 4, the test meal rats had a RIST index typical of that produced by re-feeding a solid meal thereby suggesting that HISS release was fully triggered in response to insulin following the mixed liquid meal. In contrast, the glucose rats showed only a minor change in the RIST index between the control response and the post-atropine response indicating that only a very minor, if any, release of HISS was produced by the oral glucose test.

Thus, rats fed either a solid mixed meal or a liquid mixed meal show increased insulin sensitivity secondary to release of HISS when compared to the fasted state. It appears that glucose alone is either incapable of serving as the feeding signal or the response is sufficiently transient to be essentially eliminated by the time of testing. These studies indicate that re-feeding with either a solid or liquid mixed meal is sufficient to produce a consistent restoration of HISS release and, therefore, a reversal of fasting-induced HISS-dependent insulin resistance. Thus, the classic oral glucose tolerance test is not appropriate for quantitating HDIR whereas both a solid and a liquid mixed meal causes HISS release in a manner suitable for quantitation.

### EXAMPLE THREE: DEVELOPMENT AND VALIDATION OF TEST MEAL TO QUANTITATE HISS ACTION IN HUMANS

Human volunteers were fasted overnight and submitted to a RIST. A stable glycemic baseline level was established by determining three consecutive stable venous glucose concentrations obtained at 5-minute intervals. Once a stable glycemic baseline had been demonstrated, a 50 mU/kg bolus of insulin was intravenously administered and a variable glucose infusion commenced. Arterialized venous glucose concentrations were determined at 2-minute intervals and a variable glucose infusion pump was adjusted to maintain a constant blood glucose level. After completion of the first RIST index, a standardized meal consisting of a wheat-based biscuit containing 50 grams of carbohydrate was consumed within a 10-minute period. Venous glucose measurements were determined at 5-minute intervals until a new stable baseline was obtained whereupon a second RIST was carried out.

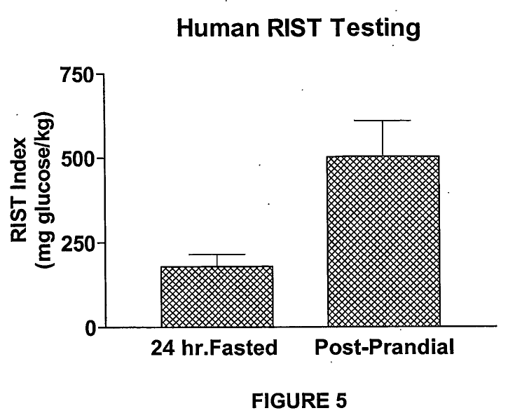

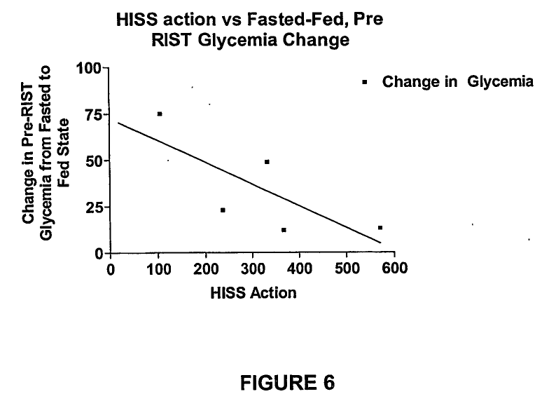

FIG. 5 shows the dynamic RIST, which represents the average from 5 subjects of the rate of glucose infusion required to maintain the glycemic target level during the RIST. The RIST index from the fasted subjects was increased dramatically following the test meal from 180 mg/kg of glucose disposal in response to the insulin bolus in the fasted stated to 502 mg/kg after the test meal. In FIG. 6, the difference between the results is plotted and is attributed to HISS action. Those subjects showing the highest HISS action, that is the greatest increase in insulin action following a test meal; also showed the least elevation in blood glucose in response to the test meal. Thus, the test meal provided a sufficient feeding signal to allow HISS release.

### REFERENCES

American Diabetes Association, Diabetes Care, Vol. 26, Supplement 1, January 2003.

Latour, M.G. and Lauft, W.W. Insulin sensitivity regulated by feeding in the conscious unrestrained rat. Can. J. Physiol. Pharmacol. 80: 8-12, 2002.

Lauft, W.W. The HISS story overview: a novel hepatic neurohumoral regional of peripheral insulin sensitivity in health and diabetes. Can. J. Physiol. Pharmacol. 77: 553-562, 1999.

Lauft, W.W. New paradigm for insulin resistance: the HISS story. In: Atherosclerosis, Hypertension and Diabetes. Eds: G.N. Pierce, M. Nagano, P. Zahradka, and N.S. Dhalla. Kluwer Academic Publishers, Boston. Chapter 21, pp. 263-276, 2003.

Reid, M.A.G., Latour, M.G., Legare, D.J., Rong, N., and Lauft, W.W. Comparison of the rapid insulin sensitivity test (RIST), the insulin tolerance test (ITT), and the hyperinsulinemic euglycemic clamp (HIEC) to measure insulin action in rats. Can. J. Physiol. Pharmacol. 80: 811-818, 2002.

Wolever, T.M.S., Chiasson, J-L., Csima, A., Hunt, J.A., Palmason, C., Ross, S.A., Ryan, E.A. Variation of postprandial plasma glucose, palatability, and symptoms associated with a standardized mixed test meal versus 75 g oral glucose. Diabetes Care 21: 336-340, 1998.

1. A method of diagnosing a metabolic disorder comprising the steps of:
(a) determining a first index value in a fasted test subject, wherein the first index value correlates with insulin action;
(b) feeding the test subject a standardized mixed meal;
(c) determining a second index value in the fed test subject, wherein the second index value correlates with insulin and hepatic insulin-sensitizing substance (HISS) action;
(d) calculating a third index value from the difference between the first and second index values; wherein the third index value correlates with HISS action; and
(e) comparing the third index value with a reference value range; wherein a metabolic disorder selected from the group consisting of insulin resistance, pre-diabetes and diabetes is diagnosed if the third index value is outside of the reference value range.

2. A method according to claim 1 wherein step (a) comprises the steps of:
taking blood samples from a fasting test subject and measuring glucose concentrations at regular intervals until the measured glucose concentrations are substantially stable;
infusing the test subject with a predetermined quantity of insulin;
taking blood samples from the insulin infused test subject and measuring the test subject's glucose concentrations at intervals;
infusing the test subject with glucose and adjusting the glucose quantity to maintain a stable glucose level, at baseline, pre-insulin levels;
determining the amount of glucose necessary to maintain euglycemia in the test subject wherein said amount of glucose establishes said first index value.

3. A method according to claim 1 or claim 2 wherein step (c) comprises the steps of:
infusing the fed test subject with a predetermined quantity of insulin;
taking blood samples from the insulin infused test subject and measuring the test subject's glucose concentrations at intervals;
infusing the test subject with glucose and adjusting the glucose quantity to maintain a stable glucose level, at baseline, pre-insulin levels; and
determining the amount of glucose necessary to maintain euglycemia in the test subject wherein said amount of glucose establishes said second index value.

4. The method according to claim 1 wherein step (a) comprises the steps of:
taking blood samples from the fasted test subject and measuring glucose concentrations at regular intervals until the measured glucose concentrations are substantially stable;
infusing the test subject with a predetermined quantity of insulin;
taking blood samples from the insulin infused test subject and measuring glucose concentrations at intervals until the measured glucose concentrations are substantially stable; and
determining a nadir value in the measured glucose concentrations of the insulin infused test subject wherein said nadir value establishes said first index value.

5. The method according to claim 1 wherein step (c) comprises the steps of:
taking blood samples from the test subject and measuring glucose concentrations at regular intervals until the measured glucose concentrations are substantially stable;
infusing the test subject with a predetermined quantity of insulin;
taking blood samples from the insulin infused test subject and measuring glucose concentrations at intervals until the measured glucose concentrations are substantially stable; and
determining a nadir in the measured glucose concentrations of the insulin infused test subject wherein said nadir value establishes said second index value.

6. The method according to claim 1 wherein step (c) comprises the steps of:
taking blood samples from the test subject and measuring glucose concentrations at regular intervals until the measured glucose concentrations are substantially stable;
infusing the test subject with a predetermined quantity of insulin; and
waiting 15 minutes and then taking a blood sample from the insulin infused test subject and measuring glucose concentrations wherein the measured glucose concentration establishes said second index value.

7. The method according to claim 1 wherein step (d) comprises the step of subtracting the first index value from the second index value value.

8. The method according to claim 1 wherein the metabolic disorder is insulin resistance.

9. The method according to claim 8 wherein the insulin resistance is hepatic insulin-sensitizing substance (HISS) dependent.

10. The method according to claim 1 wherein the metabolic disorder is pre-diabetes.

11. The method according to claim 1 wherein the metabolic disorder is diabetes.

12. The method according to claim 1 wherein the standardized mixed meal comprises carbohydrate, protein and fat.

13. The method according to claim 12 wherein the standardized mixed meal comprises 25-100 grams of carbohydrate.

14. A method of diagnosing a metabolic disorder comprising the steps of:
(a) measuring glucose concentration and hepatic insulin-sensitizing substance (HISS) concentration in a fasting test subject to provide respectively a first glucose value and a first HISS value;
(b) feeding the test subject a standardized test meal;
(c) measuring glucose concentration and HISS concentration in the fed test subject to provide respectively a second glucose value and a second HISS concentration;
(d) calculating a third glucose value using the first glucose value and the second glucose value;
(e) calculating a third HISS value using the first HISS value and the second HISS value;
(f) comparing the third glucose value with a reference glucose value range; and
(g) comparing the third HISS value with a reference HISS value range, wherein a metabolic disorder selected from the group consisting of insulin resistance, pre-diabetes, and diabetes is diagnosed if the third glucose value and the third HISS value are outside of the reference glucose value range and the reference HISS value range respectively.

15. The method according to claim 14 wherein step (d) comprises the step of:
subtracting the first glucose value from the second glucose value to establish said third glucose value.

16. The method according to claim 14, wherein step (e) comprises the step of:
subtracting the first HISS value from the second HISS value to establish said third HISS value.

17. The method according to claim 15 wherein the metabolic disorder is insulin resistance.

18. The method according to claim 17 wherein the insulin resistance is HISS dependent.

19. The method according to claim 14 wherein the metabolic disorder is pre-diabetes.

20. The method according to claim 14 wherein the metabolic disorder is diabetes.

21. The method according to claim 14 wherein the standardized test meal comprises carbohydrate, protein, and fat.

22. The method according to claim 21 wherein the standardized test meal comprises 25-100 grams of carbohydrate.

23. The method according to claim 14 wherein the measurement of HISS concentration is performed by immunoassay.

24. The method according to claim 14 wherein the measurement of HISS concentration is performed by high performance liquid chromatography.

25. The method according to claim 14 wherein the measurement of HISS concentration is performed by mass spectrometry.

26. The kit for practicing the method of claim 1 comprising a metered quantity of a standardized test meal and instructions for the method.

27. The kit according to claim 26 wherein the standardized mixed meal comprises carbohydrate, protein, and fat.

28. The kit according to claim 26 wherein the standardized mixed meal comprises 25-100 grams of carbohydrate.

29. The kit according to claim 26 further comprising a pre-measured quantity of insulin.

30. The kit according to claim 26 further comprising a pre-measured quantity of glucose.

31. The kit according to claim 26 further comprising a glucose assay kit.

32. The kit according to claim 26 further comprising a blood glucose monitor.

33. The kit according to claim 26 further comprising blood glucose paper strips.

34. The kit according to claim 26 further comprising an assay for measuring HISS kit.

35. The kit for practicing the method of claim 1 comprising a calibrated container for a standardized test meal, a glucose measuring device and instructions for the method.

36. The kit according to claim 35 further comprising a blood glucose monitor.

37. The kit according to claim 35 further comprising blood glucose paper strips.

38. The kit according to claim 35 further comprising an assay kit for measuring HISS.

39. The kit according to claim 35 further comprising alcohol swabs and lancet.
